(19) United States
(12) Patent Application Publication
(10) Pub. No.: US 2007/0185739 A1
(43) Pub. Date: Aug. 9, 2007

(54) Method and system for providing clinical care

(75) Inventors: N. Stephen Ober, Southborough, MA (US); Craig J. Piekarz, Southborough, MA (US); Joseph X. McDermott, Sudbury, MA (US)

Correspondence Address: LAW OFFICE OF DAVID H. JUDSON, 15950 DALLAS PARKWAY, SUITE 225, DALLAS, TX, 75248, US

(73) Assignee: CliniLogix, Inc., Westboro, MA (US)

(21) Appl. No.: 11/672,448

(22) Filed: Feb. 7, 2007

Related U.S. Application Data
(60) Provisional application No. 60/771,399, filed on Feb. 8, 2006.

(51) Int. Cl. G06F 19/00; A61B 5/00

(52) U.S. Cl. 705/3; 600/301

(57) ABSTRACT

The present invention describes a method that is operative within a clinical environment in which real-time locations of personnel and resources are tracked. The method begins in association with entry of a patient into the clinical environment, such as when a patient is in transit, or has been admitted, to an emergency room or to a hospital. In response, a given care guideline is identified. Using the care guideline, a set of one or more process rules, and information associated with at least one “encounter,” the system generates a patient-specific care protocol; the protocol includes a set of steps through which the patient is expected to proceed while in the clinical environment. An encounter occurs when two or more of objects (e.g., the patient, clinical personnel, and a clinical resource) are in a given physical proximity for a given time period at determined by the at least one process rule. According to the method, at least one event that occurs during at least a first step of the patient-specific care protocol is then monitored. Using information generated by the monitoring step and at least one process rule, the system then determines whether the patient moves to a next step in the patient-specific care protocol.

### CROSS-REFERENCE TO RELATED APPLICATION

This application is based on and claims priority from provisional application Ser. No. 60/771,399, filed Feb. 8, 2006.

### COPYRIGHT STATEMENT

This application also includes subject matter that is protected by copyright. All rights are reserved.

### BACKGROUND OF THE INVENTION

1. Technical Field

The present invention relates generally to providing of health care using computer, networking, and wireless technologies.

2. Background of the Related Art

Thousands of credible clinical diagnostic and treatment guidelines exist today aimed at improving patient care. The health care industry has spent decades performing clinical studies to determine optimal clinical processes and to identify the “best practices” to follow in treating many illnesses. These standards are commonly described as those derived from “evidence-based” medicine.

While these clinical recommendations are commonly accepted across the industry, implementation of and adherence to clinical guidelines is often poor. This is due to a variety of factors. Patient care often requires the coordination of many provider types offering a plurality of diverse services. This can result in confusion in selecting the right provider and service. Communication among facility staff is often poor, resulting in delays in care. Equipment and staff are frequently unavailable when needed, causing bottlenecks in the care process. Information on the location and status of requested interventions is often unavailable causing providers to waste great deals of time in locating needed staff and objects, and determining whether or not a test has been completed. These inefficiencies can result in patients frequently becoming “lost” in the process. This situation can result in poor clinical outcomes for the patient involved.

In 1999, the Institute of Medicine (IOM), a leading authority for clinical health care policy, published its land mark document “To Err is Human.” This detailed reported revealed alarming data showing that the quality of health care in the United States—especially in acute care hospitals—is fraught with errors performed by health care providers, especially physicians. It was estimated then that 93,000 U.S. citizens die each year due to human error in acute care settings. One observation of the report was that the U.S. health care systems should invest more in technologies aimed at improving patient safety and the overall quality of care. These alarming statistics have gained notice by providers, consumers, employer organizations, insurers, and the federal government. Many public and private organizations have led the way in implementing policies aimed at addressing this situation. For example, the use of bar codes on medications in hospitals is an indirect result of pressure by these groups.

The health care industry recognizes this guideline implementation problem and has established voluntary and mandated regulations to address these issues. The Joint Commission for Accreditation of Health Care Organizations (JCAHO) has developed several clinical guidelines for hospitals and other health care facilities (facility) and other care providers. JCAHO requires evidence of implementation and monitoring of these clinical guidelines during its biannual accreditation process of each US hospital. Failure of a U.S. hospital to provide evidence of its clinical guideline implementation process can result in the failure of a hospital to attain accreditation. Because the federal government requires JCAHO accreditation for U.S. hospitals to receive federal funds (including Medicare and Medicaid, which constitute approximately 40% of U.S. health care financing), failure of a hospital to attain accreditation has profound financial repercussions and may jeopardize a hospital's ability to remain solvent.

State and local governments and regulators have endorsed many of the JCAHO mandates and/or developed their own standards aimed at improving the clinical quality and outcomes of care. To enforce these recommendations, state legislatures have implemented laws and regulations mandating that facilities treating patients with certain conditions must be certified to do so by a state's department of public health or other regulatory agency. Failure to comply with these regulations results in a facility's inability to accept payment for the services rendered and/or other penalties.

### BRIEF SUMMARY OF THE INVENTION

It is an object of the invention to improve the implementation of and adherence to clinical guidelines, e.g., by providing users with a turn-key solution aimed at improving the likelihood of guideline utilization and increasing the potential of improved patient outcomes in a health care setting.

It is another object of the invention to provide the ability to automate and proactively manage the implementation of clinical care guidelines, as well as the ability to document adherence to JCAHO and other regulatory mandates.

It is still another object of the invention to provide the ability to optimize resources and assist in capacity planning in a rapid response environment, such as an emergency room of a health care facility.

A still further object is to provide the ability to automatically track the location of people and assets in real-time to improve the efficiency of deployment of such resources critical in the care process.

The system assists clinical care providers in real-time, as well as retrospectively, to maximize the possibility of providing the best care to patients. By using the system, facilities can improve clinical outcomes, decrease overall health care costs, improve patient safety, and assure regulatory compliance for their facility. Users can also optimize the utilization of resources for managing capacity and improve financial performance.

According to one embodiment, the system comprises multiple components that, when integrated, constitute a technology platform and environment that non-intrusively assists in the delivery of high quality health care. The system preferably utilizes pre-defined clinical guidelines and translates them into automated clinical “protocols” that incorporate not only a clinical “best practice” guideline, but additional information on clinical workflow that includes information on the status of critical care processes (such as patient, staff, and asset location) that often are important in the efficient implementation of clinical guidelines. The system preferably is implemented in discrete clinical “modules” by specific clinical conditions/disease state. If appropriately followed, patients on whom the system is utilized can experience a much greater likelihood of improved clinical results and a greater level of satisfaction with the overall care experience.

According to a specific feature, the invention provides the ability to identify and track the status of various clinical interventions using “encounter” logic. As used herein, an encounter is the physical proximity of two or more objects or resources (e.g., object/room, person/room, object/object, person/object, or person/person) for a specified period of time as determined by a given clinical care process algorithm.

In one embodiment, the present invention describes a method that is operative within a clinical environment in which real-time locations of personnel and resources are tracked. The method begins in association with entry of a patient into the clinical environment, such as when a patient is admitted to an emergency room or to a hospital. In response, a given care guideline is identified. Using the care guideline, a set of one or more process rules, and information associated with at least one “encounter,” the system generates a patient-specific care protocol (hereinafter referred to as a “critical care protocol” or “CCP”); the protocol includes a set of steps through which the patient is expected to proceed while in the clinical environment. An encounter occurs when two or more of objects (e.g., the patient, clinical personnel, and a clinical resource) are in a given physical proximity for a given time period at determined by the at least one process rule. According to the method, at least one event that occurs during at least a first step of the patient-specific care protocol is then monitored. Using information generated by the monitoring step and at least one process rule, the system then determines whether the patient moves to a next step in the patient-specific care protocol.

The foregoing has outlined some of the more pertinent features of the invention. These features should be construed to be merely illustrative. Many other beneficial results can be attained by applying the disclosed invention in a different manner or by modifying the invention as will be described.

### BRIEF DESCRIPTION OF THE DRAWINGS

For a more complete understanding of the present invention and the advantages thereof, reference is now made to the following descriptions taken in conjunction with the accompanying drawings, in which:

FIG. 1 is a block diagram of the invention clinical care system;

FIG. 2 is a more detailed illustration of how the CCP system is used within a health care facility;

FIG. 3 illustrates a representative object hierarchy used in the CCP system;

FIG. 4 illustrates a multiple level guideline framework that may be implemented within the CCP system;

FIG. 5 illustrates how a given CCP supports variability introduced by conditional advancement through treatment steps and facilitates compliance with the protocol;

FIG. 6 illustrates a representative CCP dashboard for the CCP for a stroke patient;

FIG. 7 illustrates representative time variables used in calculating time windows for a given CCP for the patient who presents at the facility with stroke symptoms;

FIG. 8 illustrates how a given CCP is associated with given temporal events;

FIG. 9 illustrates a timeline of the data shown in FIG. 8;

FIG. 10 illustrates how a set of encounters are generated in the CCP system;

FIG. 11 is a table describing a given interpretation of the encounters shown in FIG. 10 and how the CCP system responds; and

FIG. 12 illustrates in a more general manner how encounters are processed according to the present invention.

### DETAILED DESCRIPTION OF AN ILLUSTRATIVE EMBODIMENT

As used herein, the following terms have the following definitions:

Guideline or Clinical Guideline: A generally accepted clinical care standard developed by specialty societies, regulatory bodies, academic institutions, and/or other clinical organizations and implemented by health care providers. A Guideline typically is based on medical evidence and/or expert experience, and it is usually distributed to providers via paper and/or electronic formats. A Guideline is also known as a best practice, a critical pathway, or a standard of care.

Joint Commission for Accreditation of Health Care Organizations (JCAHO): An organization in the U.S. tasked with providing biannual accreditation to nearly every facility type. Without accreditation, facilities are unable to accept government reimbursement. JCAHO also disseminates clinical guidelines.

Clinical Care Protocol (CCP): A condition specific sequence of steps in a process of care and incorporating information derived, for example, from a Knowledge Base (as defined below), a patient record, a Workflow Engine (as defined below), a Capacity Planning Module (as defined below), and a Process Algorithm (as defined below).

CCP System (System): A system according to the present invention incorporating one or more CCPs and deployed in a given physical facility (or across a set of facilities that may be linked via a network or other communications links).

Object (or Resource): People and equipment used in a facility such as large assets, small assets, disposable products, clinical care providers, ancillary staff, support staff, and the like, and attached with a tag (e.g., an RFID tag) capable of being tracked using a reader. One or more objects are used in the diagnosis, treatment, and overall care of a patient.

Tag: A small portable device capable of transmitting and/or receiving wireless signals to and from wireless signal readers placed throughout the facility and used to locate objects, e.g., in real-time.

Context: Any information that can be used to characterize the situation of entities. A Process Algorithm uses context in part to establish “meaningful” Encounters (as defined below).

Context Awareness: The ability of the CCP system to actively establish and monitor changes in context and, using a Process Algorithm, to modify system behavior accordingly.

Encounter: The physical proximity of two or more objects or resources for a specified period of time as determined by a Process Algorithm.

Hospital (Facility) Guideline: A “facility-specific” implementation of the “JCAHO” or other recognized, national guideline as a system workflow template incorporating facility best practices within the capabilities of the resources available to that facility.

Doctor Guideline (Template): A “doctor-specific” actionable implementation of the Hospital Guideline, which may be used by the CCP system as a template for an individual doctor or a group of doctors.

Patient Plan (protocol including a doctor's orders): The actual instance of a system workflow for a specific patient episode. This is usually created from a Doctor's Template or Hospital Guideline/Protocol and can be updated dynamically as diagnosis or conditions change. The Patient Plan is the dynamic record of all pending tasks utilizing resources in the delivery of care.

Patient Record: Typically, a central database (local or remote) containing patient specific demographic and clinical information within the facility. Preferably, extracts of the database are used by the Clinical Care Protocol System. This is also known as an electronic medical record (EMR) or clinical information system (CIS).

Knowledge base: A CCP component or module containing clinical guidelines.

Workflow Engine: A CCP component comprised of databases of preferably real-time information tracking object locations within a facility.

Dashboard: A display of Process Algorithm decisions and suggested next recommended steps designed to present relevant data and information concerning a given patient's progress in the care process. The dashboard data preferably is derived from data extracts from the Patient Record, and Workflow Engine. The dashboard may be displayed on standard personal computers screens, monitors, hand-held devices, a Web browser, and/or any other interface capable of presenting visual images.

Event: Any clinical intervention on a patient during the facility admission, and a subcategory of an Encounter. An Event often is identified and classified by the identification of an Encounter. For example, a physician examining a patient in the emergency room (ER) for 15 minutes while a medication is being infused by an intravenous pump would be recognized by the CCP System as an Encounter. The components of the physician's presence and the medication infusion would constitute two clinical Events.

Episode: Typically, a single illness event experienced by a patient while admitted to a facility. An Episode usually is defined as the period of time between the patient admission date and discharge date within a facility.

CCP Trigger: Typically, any intervention performed on a patient in a facility by objects.

Capacity Planning Module (CPM): A CCP System module that projects resource utilization over time, e.g., for an entire facility or departments within a facility. CPM preferably provides real-time data on the ability of a department or object to provide a requested service based on current or future availability.

Process Algorithm (PA): A CCP system component that contains a preferably expert-derived, condition-specific rules set (one or more rules) that use clinical logic to monitor the status of clinical events (events orders, event begin time, event end time, and the like). Before proceeding to a subsequent event in a CCP, one or more criteria regarding the patient's event status typically must be satisfied. A Process Algorithm preferably uses relevant data extracted from the Patient Record, the Workflow Engine, and the CPM, and such data are analyzed, preferably in real-time, by the PA to produce a recommendation to proceed with a next planned step in the Guideline (or to otherwise recommend the completion of additional steps to satisfy the PA requirements and assure that it is appropriate to proceed with the Guideline as planned).

Reactive Mode: An implementation capability of the CCP System whereby a PA analyzes a status of an Event and determines a next appropriate Event to be performed in the process. Typically, a provider reviewing the Dashboard then uses this information to implement the subsequent Event(s) manually.

Proactive Mode: An implementation capability of the CCP System whereby the system automatically sends a series of wireless and electronic notifications to objects required to fulfill given PA criteria and to allow the patient to proceed to a next Event. Notifications include, but are not limited to, phone calls, text messages, pager notifications, and PDA alerts. Typically, the system monitors the response to the notifications and recommends advancement to a subsequent Event in the Guideline once all previous Event criteria are met. If some or all of the prior Event criteria are not fulfilled, the system may repeat the notification(s) and/or escalate the notification to an alternative object.

Retrospective Mode: An implementation capability of the CCP System whereby the system captures and analyzes all relevant data as in the other modes, but instead of displaying the results and sending notifications, the system archives these data in a structured data model for future use, e.g., in a single and/or aggregate report generation. The data in the retrospective mode can also be analyzed to identify the best clinical outcomes and link these to the best clinical care processes, thus improving the current CCPs and improving clinical outcomes.

Sentinel Event: Any Event or combination of Events deemed as the first, critical observation, typically triggering all subsequent activities and interventions on a patient. A Sentinel Event may be measured directly or estimated from manual recall.

Patient Acuity Score (PAS): An objective, data-derived measure of a patient's severity of illness, typically as determined by a set of rules embedded in a PA. Thus, for example, the PAS may be based on a patient's signs, symptoms, lab data, physician's evaluation, and other relevant data.

Facility: Any institution or sub-division of an institution providing patient care and in which the CCP System is implemented. Typically, a Facility it is an acute care hospital, although this is not a limitation. A Facility may be located in one place, or have a set of locations that are connected by communications links.

One of ordinary skill should appreciate that the nomenclature used above (as well as the use of initial capital letters for each of the defined terms) is not to be taken to limit the invention in any way. This nomenclature is provided merely for explanatory purposes. Also, the term “real-time” should be construed as a relative term given the circumstances described, and not necessarily that a given occurrence happens instantaneously. Further, one of ordinary skill should also appreciate that the examples provided throughout this written description are not intended to limit the scope of the invention, which may be used in any clinical care context to facilitate patient care within a given facility or across multiple facilities. The particular clinical care protocols (e.g., stroke) described are not meant to be taken by way of limitation either, as the invention is intended to be a general framework, architecture and methodology.

As will be seen, a CCP system 10 in FIG. 1 typically comprises multiple components that, when integrated, constitute a technology platform and environment that non-intrusively assists in the delivery of high quality health care. The system uses pre-defined clinical guidelines 12 and translates them into automated clinical “protocols” 14 that incorporate not only the clinical “best practice” guideline, but also the additional information on clinical workflow 16 that includes information on the status of critical care processes (such as patient, staff, and asset location) that are critical in the efficient implementation of clinical guidelines. As will be described, the system preferably is implemented in discrete clinical “modules” by specific clinical conditions/disease state, although this is not a requirement. If appropriately followed, patients on whom the system is utilized can experience a much greater likelihood of improved clinical results and a greater level of satisfaction with the overall care experience.

### Clinical Care Protocol System (CCP System) Description

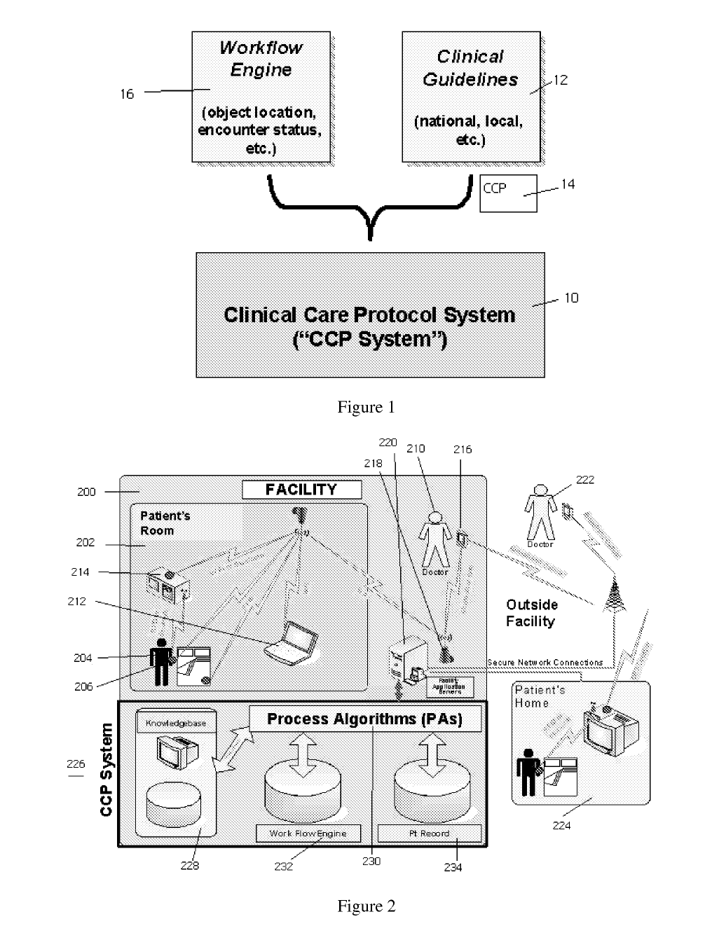

FIG. 2 illustrates an embodiment of the invention. In this example, it is assumed that the facility 200 has a number of patient rooms, including patient room 202. A patient 204 wears a tag 206 that communicates over an appropriate communications link to a reader 208. Objects within the room, such as the bed, are each also tagged, and there may be a computer 212 or other monitor or other device 214 also located in the room. A doctor 210 has a wireless or other portable device 216 that communicates to an access point 218 that is connectable to a facility application server 220. Other doctors, such as doctor 222, can communicate from outside the facility over a secure network connection. The patient's home 224 (or some object therein) may likewise be considered part of (or an adjunct to) the facility by virtue of the communication links as shown. The CCP system 226 comprises a knowledge base 228, at least one process algorithm 230, and the workflow engine 232. A patient record database 234 is shown as part of the CCP, but this is not a requirement, as the system may simply access and use pre-existing data in the facility's patient database.

The following describes an embodiment of the invention by way of an example using the components shown in FIG. 2.

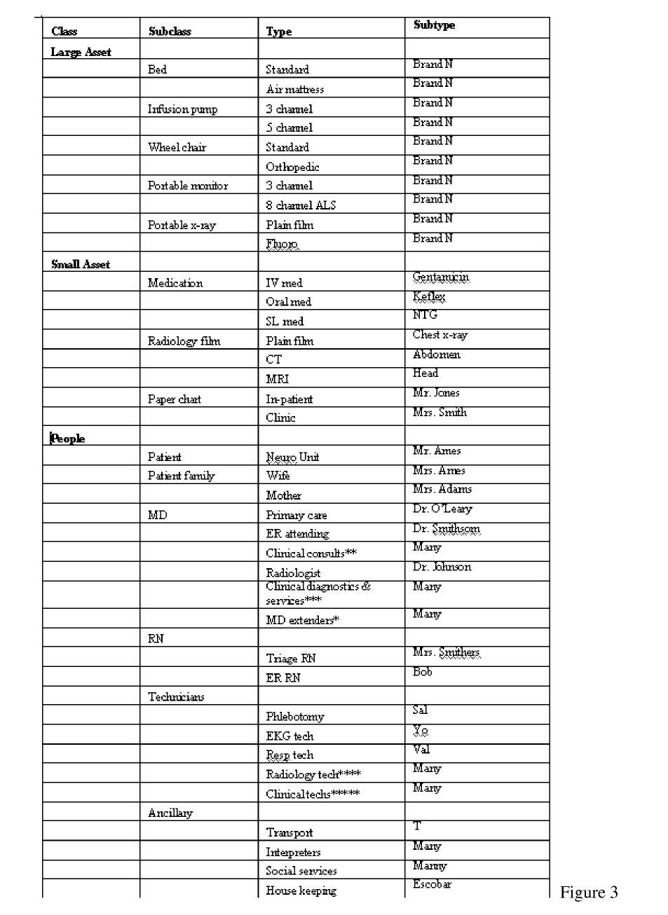

A patient (patient) entering a facility is fitted with a small portable tag (tag) capable of transmitting and/or receiving wireless signals (signals) to and from wireless signal readers (readers) placed throughout the facility. Signals include those transmitted between and among tags and readers by a plurality of wireless technologies including, but not limited to, global positioning systems (GPS), broadband cellular, radio frequency identification systems (RFID), near field communication systems (NFC), real time location systems (RTLS), wireless fidelity systems (Wi-Fi), Bluetooth, as well as infrared and bar coding systems. Other people in the facility and equipment used in the facility, such as large assets, small assets, disposable products, clinical care providers, ancillary staff, support staff, and the like (objects) used in the diagnosis, treatment, and overall care of a patient preferably are also fitted with a tag similar to that worn by each patient. Preferably, the patient tag contains a unique patient identifier that wirelessly links each patient with demographic and clinical information about the patient contained in a central database (i.e., a patient record database) stored within the facility. The object tag preferably contains a unique object identifier that wirelessly links each object with descriptive information about the object contained in a workflow engine within the facility. The system preferably also categorizes objects into a hierarchical arrangement comprising class, subclass, type, and subtypes. A table shown in FIG. 3 is representative of this hierarchical arrangement. The tag/reader combination preferably transmits the exact location of the patient and all tagged objects in the facility to a central location database periodically, e.g., every few minutes or seconds. As used herein, preferably the “facility” includes areas adjacent to the facility and areas commonly identified as part of the facility, such as parking lots, walkways, common outside areas, and the like. In addition to connectivity within the facility, and as illustrated in FIG. 2, the system has the capability of establishing connectivity anywhere outside the facility using long distance and global wireless communications technologies. These technologies are mostly used for paging/contacting providers and transmitting clinical data on patients from their homes. Preferably, readers are strategically placed in the ceilings, doorways, and other locations throughout the facility to assure an uninterrupted signal between the tags and readers, assuring the most accurate identification of patient and object location.

In addition to capturing detailed data of a patient's experience within the facility, the patient record preferably contains the reason(s) a patient is admitted to or seeking treatment at the facility for a particular illness (episode). If a patient meets specific clinical criteria contained in the guideline knowledge base (knowledge base), a CCP trigger is initiated that flags the particular patient as a candidate for inclusion in activating a specific system protocol. Preferably, activation is based on the patient's signs, symptoms, and medical history. The system preferably utilizes industry standard scripts to accurately identify and extract specific data in the patient record that may be required to trigger the system. Examples of industry standards in this area include those under development by SAGE, a collaborative project involving several academic and industry organizations to create data extraction standards to better implement a guideline deployment model. Preferably, guidelines in the knowledge base are specific to a particular disease, condition, or injury such as stroke, heart attack, drug overdose, respiratory disease, and the like, and they are often targeted toward clinical states where the timely/swift implementation of diagnostic and/or therapeutic interventions is critical to assure a favorable clinical outcome. Preferably, guidelines are selected, developed and maintained by the user of the system. In one embodiment, the system allows users to initially select from a series of nationally recognized guidelines that can be customized by users to reflect their own local care standards. The customized guidelines may be quickly updated as needed by the user. New guidelines can be easily added on a regular or ad-hoc basis.

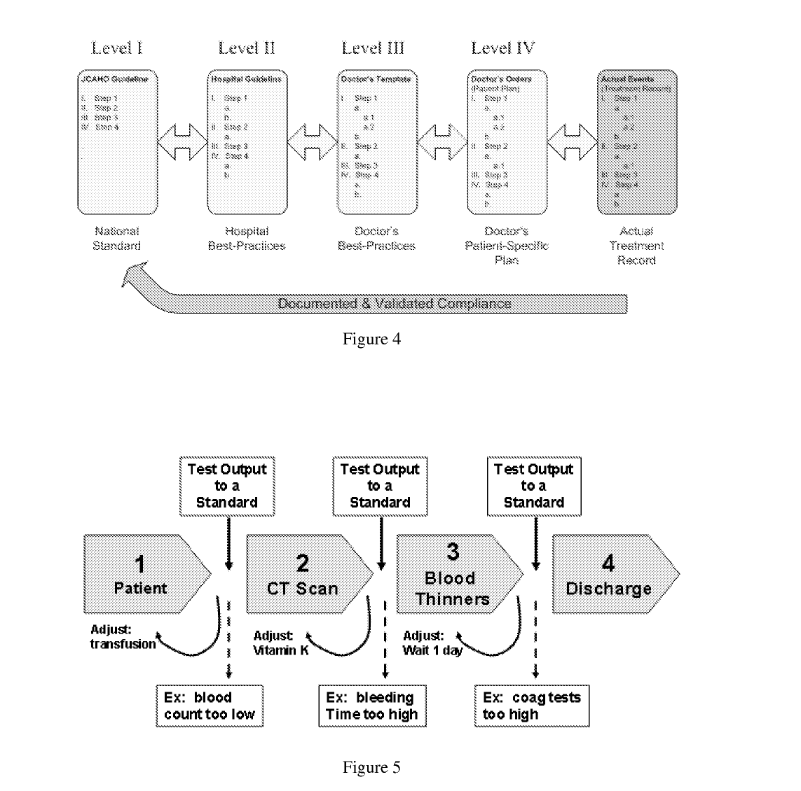

A multi-level guideline architecture employed by the CCP ensures that actionable workflows can be implemented within higher-level frameworks to facilitate compliance and the validation thereof. An example of a multi-level guideline according to the invention is shown in FIG. 4. In this example, four levels are utilized, and this framework comprises a JCAHO guideline at the highest Level I, followed by a hospital guideline (Level II), which is followed by a doctor's template (Level III), which in turn is followed by a doctor's orders (Level IV). The actual events then form an actual treatment record for the patient.

According to a feature of the invention, preferably the CCP system comprises one or more process algorithms. As noted above, a process algorithm typically is an expert-derived, condition-specific rules set (comprising one or more rules) that uses clinical logic to monitor when clinical interventions (events) have been requested, begun, ended, and the like. Before proceeding to a subsequent event in the PA, one or more criteria regarding a patient's event status typically must be satisfied. A process algorithm typically uses relevant data extracted from one or more of: the patient record, the workflow engine, and a capacity planning module (CPM), and such data are analyzed in real-time by the algorithm to produce a recommendation to proceed with the next planned step in the guideline or, in the alternative, to complete additional steps to satisfy a PA requirement that it is appropriate to proceed with the guideline as planned. This is best illustrated by way of example.

In particular, prior to transporting a patient from the emergency room (ER) to the radiology department to receive an x-ray test, the x-ray machine must be available to accept the patient (has appropriate capacity) and a radiology technician must be available near the x-ray machine to perform the procedure. In the case that the x-ray equipment and/or the technician are unavailable, a given process algorithm in the system automatically notifies the appropriate personnel and recommends that the patient remain in the ER to be better monitored until the radiology department is prepared to service the patient. In this example, the process algorithm determines when it should intervene and notify personnel, preferably based on specific timetables within the algorithm that support the time standards in the clinical guideline relevant to this event. As another PA example, prior to giving a patient a blood clot busting drug to treat a life threatening stroke, the patient must have the results of coagulations (bleeding) studies available to assure that the patient will not have major bleeding side effects when the drug is administered. If the system does not have this data available, the system will automatically notify the laboratory and other staffs to assure that these data are available prior to proceeding with the administration of the clot busting medication. Once again, the particular process algorithm determines when it should intervene and notify the laboratory based on specific timetables within the algorithm that support the time standards in the clinical guideline relevant to this event.

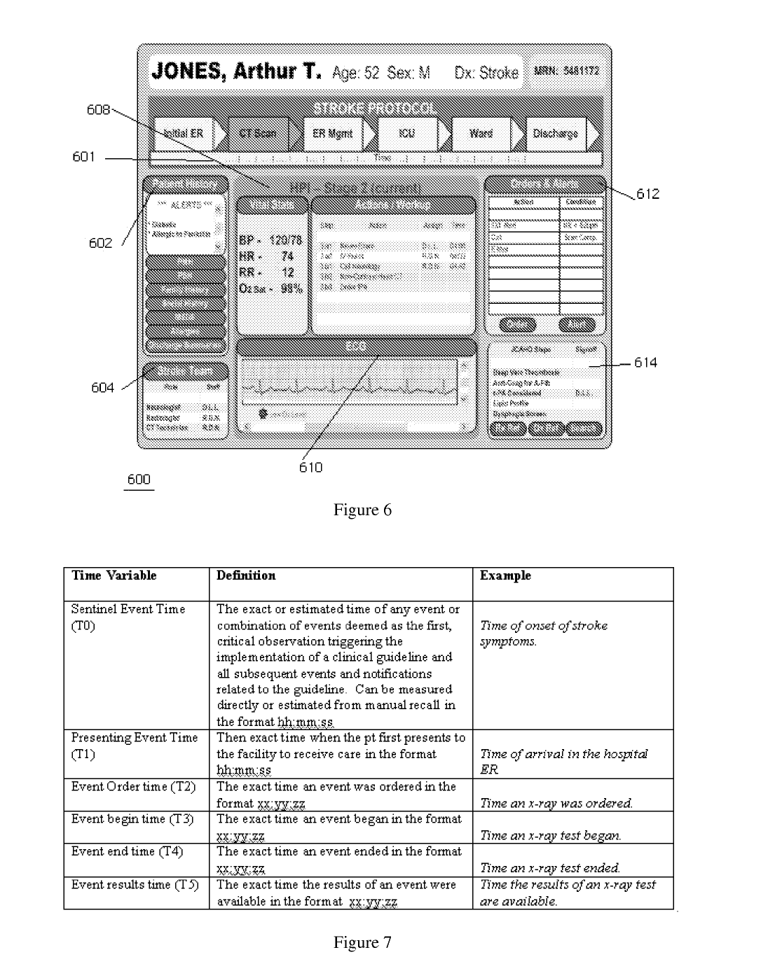

A given CCP supports the variability introduced by conditional advancement through treatment steps and facilitates compliance with the protocol. This is illustrated in FIG. 5. In this example, a patient's blood is taken and tested against a given standard. The blood count has been found to be too low, necessitating a CT scan. The result of that scan is again tested against a standard, at which point the system has determined that the patient's bleeding time is too high. The CCP dictates the patient be put on a blood thinner. After a subsequent test, the blood thinning regimen may be altered or, if complete, the patient is discharged. Throughout the CCP, preferably the PA decisions and suggested next recommended step(s) are displayed on a CCP dashboard 600, such as illustrated in FIG. 6. As can be seen, preferably the dashboard provides a convenient CCP system interface designed to present relevant data and information concerning the patient's progress in the care process. Typically, the dashboard data is derived from data extracts from the patient record, and the workflow engine. In this example, the CCP is a stroke protocol, which includes the treatment phases (as indicated by display tab) Initial ER, CT Scan, ER Management, ICU, Ward, Discharge, as illustrated. When the operator selects a given display tab (in this case CT Scan), the details of the given phase are displayed in detail. As can be seen, the dashboard also includes a timeline 601, relevant patient history tabs 602 (which may include, for example, patient history, family history, social history, allergies, medications, and the like), relevant treating personnel 604, given clinical data of the actual test 606, other relevant clinical data 608, orders and alerts 610, and other information 612 (such as the JCAHO protocol). As noted above, the dashboard may be displayed on a standard personal computer screen, a monitor, a handheld device, a web browser, and/or any other interface capable of presenting visual images.

The PA decisions can be implemented in one or more modes: Reactive, Proactive and Retrospective. In a Reactive mode, the PA analyzes the status of an event and determines the next appropriate event in the process. A provider reviewing the dashboard then uses this information to implement the subsequent event(s) manually. In a Proactive mode, the system automatically sends a series of one or more wireless and/or electronic notifications to objects required to fulfill the PA criteria and to allow the patient to proceed to the next event. Notifications include, but are not limited to, phone calls, text messages, pager notifications, and PDA alerts. In the Proactive mode, the system preferably monitors the response to the notifications and recommends advancement to a subsequent event in the guideline once all previous event criteria are met. If some or all of the prior event criteria are not fulfilled, the system may repeat the notification(s) and/or escalate the notification to an alternative object. For example, if no x-ray technician is available in the x-ray department for a patient to receive his or her x-ray, the x-ray technician on duty is automatically paged. Failure of the technician to respond within a pre-set time interval (in this example) may initiate a second notification. This time, however, the technician's supervisor may also be alerted of the problem and the need for an immediate response. In a Retrospective mode, the system preferably captures and analyzes all relevant data as in the other modes, but instead of displaying the results and sending notifications, the system archives such data (preferably in a structured data model) for future use (e.g., in single and/or aggregate report generation). In the Retrospective mode, clinical performance can be measured across the facility to determine the adherence to clinical guidelines for the entire facility or group of facilities. Reports may be generated to monitor the performance of groups of providers, various clinical departments, and individuals. The data in the Retrospective mode can also be analyzed to identify the best clinical outcomes and link these to the best clinical care processes, thus improving the current CCPs and improving clinical outcomes. For example, observing that patients receiving a certain medication within one hour of arrival in the ER with symptoms of stroke had better outcomes than those patients receiving the medication after one hour could result in the development of a new standard of care for patients suffering from stroke. The archived data can also be used to identify potential candidates for clinical studies.

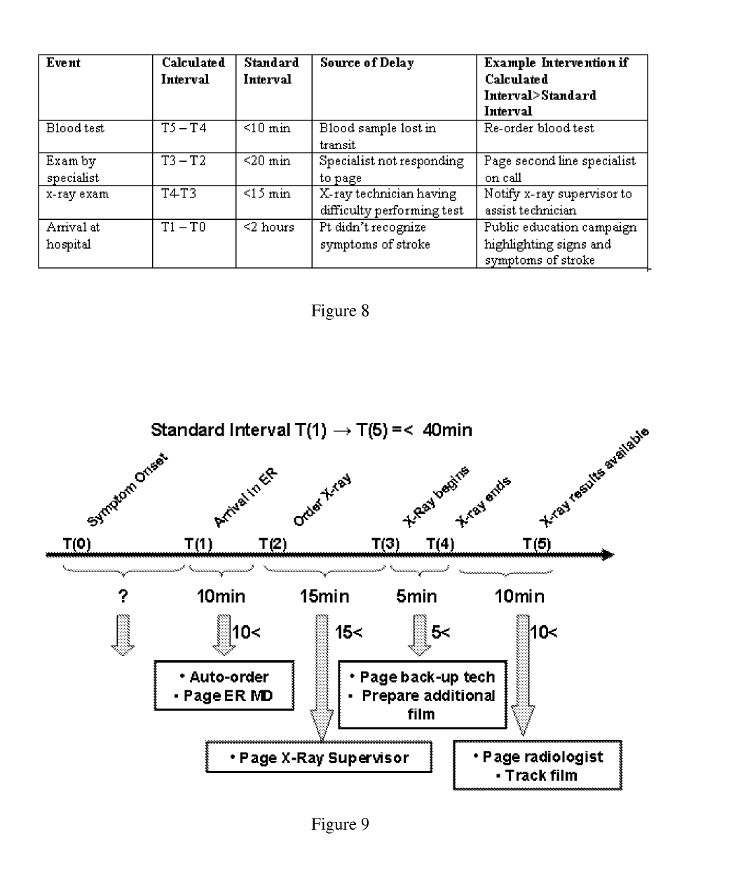

According to a feature of the invention, as noted above preferably each tagged patient and object transmits location information on a regular basis to the CCP workflow engine. Each signal preferably is time stamped to determine the exact time and location of each patient and object. In addition to absolute (or relative) time/location recordings, the CCP system preferably includes a series of timing elements used to measure the duration of specific events. Each protocol preferably has at least one sentinel event that serves as the beginning of a patient's episode of illness. Preferably, each subsequent event is measured in terms of length of time from the sentinel event. In addition to length of time between the sentinel event and subsequent events in the care process, several other time duration windows preferably are calculated and that may be used in assuring adherence to the clinical guideline. The table shown in FIG. 7 shows representative time variables used in calculating time windows for a given CCP for stroke. By calculating the length of time between certain time variables and known capacity constraints, the CCP system automatically identifies potential delays in the care process and takes appropriate actions, such as alerting individuals (people objects) to intervene in the process via electronic notification. The system then determines when to intervene by comparing the calculated time intervals to standard intervals embedded in the clinical guideline. FIG. 8 illustrates a representative table generated by this process, with the resulting data displayed as a timeline in FIG. 9. These are merely representative examples of the CCP system (and a given CCP) evaluates and uses temporal (as well as spatial, such as location) considerations.

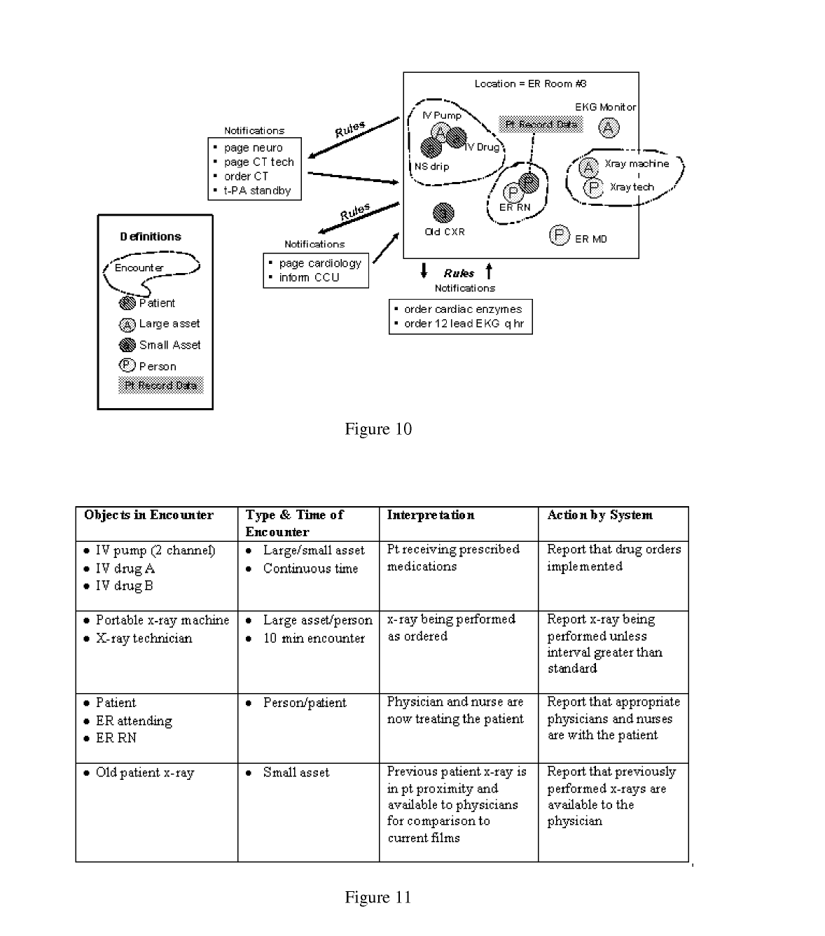

According to another feature of the invention, in addition to monitoring the specific location of objects, the system also identifies encounters. As previously noted, encounters are a type of often complex event used in determining the adherence to clinical guideline criteria; a given encounter typically is defined by the juxtaposition of two or more objects for a predetermined period of time. Based on the object type and duration of the encounter, preferably each encounter is classified into a specific type that has clinical relevance and meaning in determining the fulfillment or initiation of the suggested event by the PA. A diagram of several ER encounters is shown in FIG. 10. In this example, the patient (fitted with a tag) is in ER #3, which includes a number of large assets including an IV pump, an EKG monitor, and an x-ray machine. The IV pump has a number of small assets associated therewith, such as an IV drug. In addition to the patient, an ER nurse is seen to be present, as well as an x-ray technician and an emergency room physician. The patient record data is accessible to the system also indicated. The system includes the set of one or more rules embedded in one or more process algorithms based on the expert analysis of the encounter data. By analyzing the encounter data, the system determines the most appropriate response and intervention. Several examples of encounter types (corresponding to the example in FIG. 10) and CCP system responses appear in the table of FIG. 11 by way of example only.

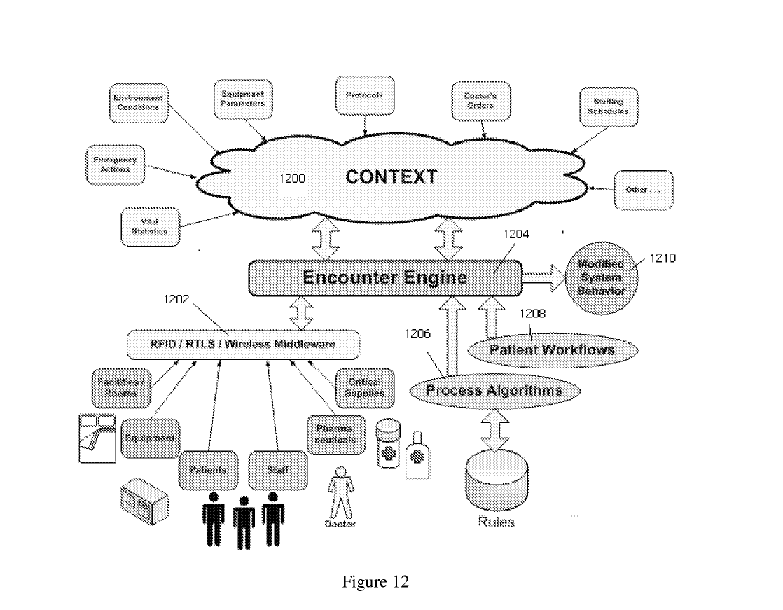

FIG. 12 illustrates the encounter functionality in a more general manner. As can be seen, a given context 1200 is defined by one or more of the following factors and associated data: vital statistics, emergency actions, environment conditions, equipment parameters, protocols, doctor's orders, staffing schedules, and the like. The workflow engine is also aware of the various physical assets, which includes facilities/rooms, equipment, patients, staff, pharmaceuticals and critical supplies, and such location information is made available by the real-time location system (RTLS)/wireless middleware layer 1202. The context data and the location data is provided to the encounter engine 1204, which also receives the process algorithm(s) and patient workflow(s). In response, the encounter engine 1204 modifies system behavior as required to meet the desired objective. Thus, in the example above, the encounter engine uses context awareness and process algorithms to establish “meaningful encounters,” and it dynamically modifies system behavior accordingly. As already noted, the Process Algorithms 1206 define the complex combinations of resources, context and temporal parameters required to establish a meaningful encounter. The Protocols, as well as the Patient Workflows 1208, establish the collection of anticipated encounters and critical timeframes that the system proactively monitors. Changes in the locations and proximities of resources reported to the system by the wireless Real Time Locator System (RTLS) preferably are continuously evaluated by the encounter engine which dynamically updates the context and drives modified system behavior 1210.

One of ordinary skill will recognize that the volume of data required to efficiently plan the complex combination of resources and tasks involved in multiple patient care cycles often is too burdensome to be entered manually on a timely basis. By using the encounter engine, the system dynamically modifies the user interface based on changing conditions and anticipated usage patterns. In particular, by maintaining a state of context awareness, the system can respond from a centralized or server-based perspective, or on a local, device-specific basis. These facilitated usage capabilities make the CCP system unique and easy to use, addressing one of the most critical obstacles to adoption and disciplined use of the system. An example of a CCP facilitated user experience (a Doctor doing rounds) is as follows. This is merely representative.

A Doctor selects a generic tablet pc from the charging stand at the nurse's station to do rounds. By recognizing the encounter between the doctor and computing device, the tablet automatically prompts the Doctor (by name) to touch a fingerprint scanner (or enter a password) to log in. Automatically, the tablet identifies and validates the doctor and retrieves this doctor's list of patients on this particular floor, in this particular wing—because the system knows where it is and where the doctor and patients are. Now, assume that the Doctor walks into room 1027 with 2 patients—A & B, and that this Doctor has been assigned to Patient A but not to Patient B. The system automatically retrieves the record for Patient A (anticipating the encounter), then validates the encounter through proximity. It then prompts the doctor, for example: “Do you wish to see Patient A's records?” Upon indicating yes, the relevant data is presented (preferably in the Doctor's personal and pre-defined format) that may include patient history, notes, names of spouses, children or siblings, and the like. Now, assume that the Doctor is paged; he or she the puts down the tablet pc and then walks away. In this example, the encounter engine recognizes that the encounter has been terminated or interrupted, and the tablet immediately locks and logs the Doctor off so that no private information can be compromised.

Capacity Availability and Planning

CCP can maintain the definition of all of the hospital resources involved in the health-care continuum. As used herein, a resource is a broad term used to describe anything that might be involved in fulfilling a doctor's order or hospital compliance requirement. These include but are not limited to: facilities/rooms, equipment, people, pharmaceuticals and supplies.

Static Resource Capacity and Capability Model

The definition of these resources preferably includes a detailed description of the resource capabilities and constraints to enable the system to differentiate them in a planning scenario. These capabilities may include, for example, certifications for staff, bed-capacity for a room, and the like. Constraints may include information such as weight limits, size limits, and the like. With this information, a static capacity and capability model can be built.

Dynamic Resource Capacity and Capability Model

Process algorithms managed by the system, combined with experiential data, may be used to create facility-wide and department level capacity models. Projecting resource utilization over time, the system can determine the availability of various object types and facility departments. A Capacity Planning Module (CPM) provides real time data on the ability of a department or object to provide a requested service based on current or future availability. The system's auto-identifying and auto-locating technologies confirm the presence of planned resources and issues alerts when discrepancies are found. Thus, for example, at the beginning of each employee shift in facility departments directly or indirectly involved in patient care (e.g., laboratories, radiology, operating rooms, post-anesthesia care units, intensive care units, and the like), the CPM may prompt each department supervisor to confirm or edit information about the department's capacity and ability to service patients during that shift. While each department is typically assigned a standard patient load capacity per day, this capacity can dramatically change based on daily department staffing levels and severity of illness of patients being evaluated in that department (more severely ill patients often require longer evaluation periods). A clinical guideline may require a patient evaluation in a new department (e.g., radiology) from where the patient is currently located (ER). The system checks the capacity of the department of interest to accept or service the patient. Preferably, the CPM contains scheduling data for each facility department as well as an acuity score for each patient that is used to establish the priority in which each patient will receive services. The patient acuity score preferably is based on an evaluation of a patient's severity of illness as determined by a set of rules embedded in the PA. Thus, for example, the patient acuity score (PAS) may be based on a patient's signs, symptoms, lab data, and physician's evaluation.

Preferably, the CPM tracks passage of time, and it contains or enforces given standards, such as unacceptable wait times based upon the potential for future clinical complications if the requested service is not delivered as scheduled (e.g., x-ray not performed on time). In a representative embodiment, a given PA determines how quickly a patient with a certain condition requires evaluation by each requested department. The system then uses the patient acuity score to re-prioritize its patient evaluation schedule within each department to ensure that patients of higher acuity (most severely ill) receive the most preferential requested departmental service at the time required. Patients receiving services for non-urgent elective procedures may be re-prioritized to times other than those originally scheduled. Typically, all high acuity score patients receive preferential services. However, once information is received concerning a department service request for a high acuity patient, the system preferably automatically alerts the department staff of the situation and recommends appointment cancellations, rescheduling, and re-prioritization options to assure that the high acuity patient receives the requested service as quickly as possible.

The CPM may also take into account department specific issues that may impact a department's ability to service patients. These may include equipment failures, planned equipment maintenance and calibration, long-term and short-term staffing shortages, and the like. This department specific information is maintained by the CPM to assure that each department is able to service an appropriate number of patients during each subsequent work shift. Preferably, schedules and available capacity are maintained and viewable on a patient-specific, department-specific, or facility-wide basis. This enables the hospital to make admission decisions on a condition-specific basis, taking into account issues of downstream capacity rather than the relatively easily observed full capacity situation that may exist at the point of patient facility entry, such as manifest by acute ER overcrowding. For example, and ER full of patients with broken limbs and the flu (but an open cardiac ward) may cause a hospital to divert a cardiac patient to another hospital. If the receiving hospital has an empty ER, but a full cardiac ward, the facility may still choose to divert cardiac patients to other facilities do to its inability to provide appropriate cardiac service after evaluation in the ER.

By identifying critical resource constraints, system data also may suggest that a patient in transit be re-routed to another facility.

Dynamic Resource Supply and Demand

The system preferably tracks the dynamic supply and demand on each of the resources involved in planning. As is well known, supply and demand come from a variety of different sources, and in both predictable and unpredictable ways. The CCP system preferably uses staffing schedules, OR schedules, routine equipment maintenance and calibration schedules, and any other source of planned supply and demand, to facilitate a rough-cut scheduling of resources. As noted above, the CCP system then handles the variable and dynamic nature of the clinical environment by accommodating and adapting to changing conditions and doctor's orders.

The system can also be used to automatically establish the status of a resource for planning and optimization purposes. One of the critical inputs to any resource planning and optimization system is the status of a given resource (e.g., available, unavailable, undergoing maintenance, or the like). A simple example is the IV pump in the example ER encounter scenario described above. By combining the context data with patient workflows, the CCP can establish the difference between pumps that are definitely not available, from those that are potentially available, from those that are definitely available. Thus, for example, a pump in a room with a patient in the room and with order for IV is not available. However, if the encounter establishes a pump in the room with a patient not in the room and with no pending order for IV, the pump is potentially available. In yet another alternative, if the pump is in a room of a patient that has been discharged, it is definitely available. Thus, the ability to automatically establish the status of a resource for planning and optimization purposes provides another advantage of the CCP system. Of course, this methodology can be expanded. Thus, continuing with this example, the room of a discharged patient is “unoccupied,” but not “available” until the system detects the encounter of a “cleaning person” in the room for a given time.

### Implementing Technologies

The present invention may be implemented with any known or later-developed wireless and computer networking technologies. Thus, for example, the wireless infrastructure illustrated above may include any wireless client device, e.g., a cell phone, pager, a personal digital assistant (PDA, e.g., with GPRS NIC), a mobile computer with a smart phone client, or the like. A typical mobile device is a wireless access protocol (WAP)-enabled device that is capable of sending and receiving data in a wireless manner using the wireless application protocol. The wireless application protocol (“WAP”) allows users to access information via wireless devices, such as mobile phones, pagers, two-way radios, communicators, and the like. WAP supports wireless networks, including CDPD, CDMA, GSM, PDC, PHS, TDMA, FLEX, ReFLEX, iDEN, TETRA, DECT, DataTAC, and Mobitex, and it operates with many handheld device operating systems, such as PalmOS, EPOC, Windows CE, FLEXOS,OS/9, and JavaOS. Typically, WAP enabled devices use graphical displays and can access the Internet (or other communication network) on so-called mini- or micro-browsers, which are web browsers with small file sizes that can accommodate the reduced memory constraints of handheld devices and the low-bandwidth constraints of a wireless networks. In addition to a conventional voice communication, a given mobile device can communicate with another such device via many different types of message transfer techniques including SMS (short message service), enhanced SMS (EMS), multi-media message (MMS), e-mail WAP, paging, or other known or later-developed wireless formats.

Any known or later developed RFID technologies may be used for the tag and readers. As is well-known, radio frequency identification (RFID) is an automatic identification method that relies on storing and remotely retrieving data using devices called RFID tags or transponders. As used herein, an RFID tag is an object that can be attached to or incorporated into a product or person for the purpose of identification using radio waves. The RFID tags may be active (internally powered) or passive (powered by the received RF energy). Any commercial RFID tags and RFID systems for workflow and inventory management may be used for this purpose.

The other components illustrated comprise a set of one or more computing-related entities (systems, machines, process programs, libraries, functions or the like) that together facilitate or provide the inventive functionality described. In a typical implementation, the infrastructure comprises a set of one or more computers. A representative machine is a network-based server running commodity (e.g. Pentium-class) hardware, an operating system (e.g., Linux, Windows, OS-X, or the like), an application runtime environment (e.g., Java, ASP) and a set of applications or processes (e.g., Java applets or servlets, linkable libraries, native code, or the like, depending on platform), that provide the functionality of a given system or subsystem. The service may be implemented in a standalone server, or across a distributed set of machines.

Typically, a server connects to the publicly-routable Internet, a corporate intranet, a private network, or any combination thereof, depending on the desired implementation environment. Of course, any other hardware, software, systems, devices and the like may be used. More generally, the present invention may be implemented with any collection of autonomous computers (together with their associated software, systems, protocols and techniques) linked by a network or networks.

As previously noted, the hardware and software systems in which the invention is illustrated are merely representative. The invention may be practiced, typically in software, on one or more machines. Generalizing, a machine typically comprises commodity hardware and software, storage (e.g., disks, disk arrays, and the like) and memory (RAM, ROM, and the like).

The particular machines used in the network are not a limitation of the present invention. A given machine includes network interfaces and software to connect the machine to a network in the usual manner.

While given components of the system have been described separately, one of ordinary skill will appreciate that some of the functions may be combined or shared in given instructions, program sequences, code portions, and the like.

While the above describes a particular order of operations performed by certain embodiments of the invention, it should be understood that such order is exemplary, as alternative embodiments may perform the operations in a different order, combine certain operations, overlap certain operations, or the like. References in the specification to a given embodiment indicate that the embodiment described may include a particular feature, structure, or characteristic, but every embodiment may not necessarily include the particular feature, structure, or characteristic.

Having described our invention, what we now claim is as follows.

1. A method, operative within a clinical environment in which real-time locations of personnel and resources are tracked, comprising:
in association with entry of a patient into the clinical environment, identifying a care guideline;
using the care guideline, a set of one or more process rules, and information associated with at least one encounter to generate a patient-specific care protocol having a set of steps through which the patient is expected to proceed while in the clinical environment, wherein an encounter occurs when two or more of the following objects are in a given physical proximity for a given time period at determined by the at least one process rule: the patient, clinical personnel, and a clinical resource;
monitoring at least one event that occurs during at least a first step of the patient-specific care protocol and using information generated by the monitoring step and at least one process rule to determine whether the patient moves to a next step in the patient-specific care protocol.

2. The method as described in claim 1 wherein the care guideline is one of: a guideline created by a regulatory entity, a guideline associated with the clinical environment, and a guidelines associated with a given treating physician.

3. The method as described in claim 1 wherein data derived from a patient record also is used to facilitate generation of the patient-specific care protocol.

4. The method as described in claim 3 wherein resource availability data also is used to facilitate generation of the patient-specific care protocol.

5. The method as described in claim 4 wherein the resource availability data determines availability of a given resource or set of resources at a given time.

6. The method as described in claim 1 further including displaying given information associated with the patient-specific care protocol.

7. The method as described in claim 6 wherein the given information includes information associated with a given step of the patient-specific care protocol.

8. The method as described in claim 1 further including issuing at least one notification as a result of the monitoring step.

9. The method as described in claim 8 further including determining whether a given response is received to the notification and, if so, taking a given action.

10. The method as described in claim 9 wherein the given action is generating a recommendation to advance the patient to the next step in the patient-specific care protocol.

11. The method as described in claim 1 wherein the clinical environment is located in a given health care facility and the real-time locations of personnel and resources are tracked using at least radio frequency identification.

12. A method, operative within a health care facility in which real-time locations of personnel and resources are tracked and resource availability is known, comprising:
using a care guideline, a set of one or more process rules, resource availability data, and information associated with at least one encounter to generate a care protocol having at least first and second steps through which a patient is expected to proceed while in the clinical environment, wherein an encounter occurs when two or more of the following objects are in a given physical proximity for a given time period at determined by the at least one process rule: the patient, clinical personnel, and a clinical resource;
evaluating a context during the first step of the care protocol to determine whether the patient moves to the second step in the care protocol.

13. The method as described in claim 12 wherein the care guideline is one of: a guideline created by a regulatory entity, a guideline associated with the clinical environment, and a guidelines associated with a given treating physician.

14. The method as described in claim 12 wherein data derived from a patient record also is used to generate the care protocol.

15. The method as described in claim 12 further including displaying given information associated with each of the first and second steps of the care protocol.

16. The method as described in claim 12 further including issuing at least one notification as a result of evaluating the context.

17. The method as described in claim 16 further including determining whether a given response is received to the notification and, if so, taking a given action.

18. The method as described in claim 12 wherein the real-time locations of personnel and resources are tracked using at least radio frequency identification.

19. A method, operative within a health care facility, comprising:
tracking real-time locations of personnel and resources to generate real-time encounter data, wherein the encounter data defines one or more encounters, and wherein an encounter occurs when two or more of the following objects are in a given physical proximity for a given time period: a patient, clinical personnel, and a clinical resource;
evaluating the encounter data against given context data; and
modifying a given system behavior as a result of the evaluation.

20. The method as described in claim 19 wherein the given context data is one of: a vital statistic, an emergency action, an environment condition, an equipment parameter, a protocol, a treatment order, and a staffing schedule.
